(19) United States
(12) Patent Application Publication
(10) Pub. No.: US 2021/0287784 A1
(43) Pub. Date: Sep. 16, 2021

(54) WIRELESS CHECK-IN SYSTEM FOR HEALTHCARE ENVIRONMENTS

(71) Applicant: Quedo Inc., Minnetrista, MN (US)

(72) Inventor: Robert Mullaney, Minnetrista, MN (US)

(21) Appl. No.: 17/202,293

(22) Filed: Mar. 15, 2021

Related U.S. Application Data
(60) Provisional application No. 62/990,292, filed on Mar. 16, 2020.

(51) Int. Cl. G16H 40/20; G16H 10/60; G06Q 10/10; G16H 10/20; G06Q 50/22; H04L 29/06; H04W 4/021

(52) U.S. Cl. CPC G16H 40/20; G16H 10/60; G06Q 10/10; H04W 4/021; G06Q 50/22; G06Q 10/1095; H04L 63/04; G16H 10/20

(57) ABSTRACT

A wireless check-in system provides the coordination between a patient application operating on a mobile device, and systems managed or operated by a healthcare provider to allow for check-in steps to be achieved remotely. In this manner, typical interaction with a front desk is avoided or greatly minimized. Further, all information needed for effective patient care can be obtained prior to arriving at the healthcare provider facility. A healthcare provider will also have real-time access to a tracking board which will show the location and status or patients waiting for appointments, allowing for effective coordination.

### BACKGROUND

Patients seeking healthcare services typically encounter a reception or check-in desk when entering a clinic, hospital, or other healthcare facility. In these settings, front desk staff will typically collect information about the patient (i.e. name, address, insurance information, purpose for visit, etc.) by having the patient fill out appropriate forms or by obtaining copies of necessary paperwork. Inevitably, this process will involve the passing of papers, pencils/pens, cards, etc. between the patient and the front desk staffer. Once appropriate information is obtained, the patient is then directed to an appropriate waiting location, or to additional staff people.

This existing process is problematic for a number of reasons: (i) the patient is confronted with an unwelcoming and “cold” check-in desk upon first entering the facility; (ii) it is awkward and unsettling for the patient when entering the facility, (iii) it often takes many questions and a timely exchange for the front desk staff to obtain the required amounts of information (which could be misinterpreted or misconstrued), and (iv) the passing of physical items between the front desk staff and the patient presents an opportunity for germs and viruses to be exchanged. Although these existing processes and procedures allow the healthcare providers to obtain the information they feel they need, this approach is not friendly or efficient.

### SUMMARY

In an effort to provide safety, efficiency and improved effectiveness for healthcare facilities, an automated check-in system provides the tools for a patient to remotely check-in for appointments in a manner that allows them to avoid or minimize their contact with the traditional “front desk”. Once check-in is achieved and the provider is ready, the patient can enter the facility with appropriate instructions and capabilities to access appropriate areas. This process is achieved by through the coordination of various components, thus creating a system that is efficient and easy to use.

### BRIEF DESCRIPTION OF THE DRAWINGS

The details of the wireless check-in system will be better understood by reading the following description which presents certain details related to one embodiment of the wireless check-in system, in conjunction with the drawing, in which:

FIG. 1 is a schematic diagram showing various components of the disclosed embodiment of a wireless check-in system;

FIG. 2 illustrates a welcome/sign-in screen;

FIG. 3 shows a profile entry page;

FIGS. 4-6 present ID card capture features;

FIGS. 7-8 show appointment scheduling tools;

FIG. 9 illustrates upcoming appointments presented to a user;

FIG. 10 presents an appointment status screen with “checked-in” status presented;

FIG. 11 shows a messaging utility;

FIG. 12 illustrates a questionnaire process;

FIG. 13 presents a questionnaire confirmation screen;

FIG. 14 illustrates a patient status map viewable by the healthcare provider;

FIG. 15 presents an illustration of the geofencing capability of the wireless check-in system;

FIG. 16 shows a 2D representation of a patient status map; and

FIG. 17 provides one possible layout for a facility entry area and further presents the automated access capabilities.

### DESCRIPTION

To provide patient comfort and addition efficiency, the wireless check-in system described below includes a software platform that exploits the features of several separate software applications to automate the check-in process usable by patients at a healthcare facility. By coordinating the operations of the various application in a manner to provide efficient interaction and collection of information, a powerful system is realized which has multiple benefits and advantages.

At a high level, the embodiments of the wireless check-in system set forth below comprises a GPS enabled application running on a mobile device, a facility system (operating at the particular healthcare facility, or coordinated by the healthcare facility), and a back-end system operating at a data center or similar computing facility. Each of the systems are configured to share information and trigger specific processes when particular conditions are met. It should be recognized that the actual configuration of the above-mentioned systems or applications can vary. For example, the back-end system and facility system could be combined and operate on a single server or computer.

In the discussion below, certain features are described in the context of a patient encounter with a healthcare provider. It is understood that similar systems could be used for any type of user and is not limited to use by patients and healthcare environments. That said, the features and advantages are particularly beneficial for patient check-in at healthcare facilities.

It is generally contemplated that the overall check-in system will be made available and managed by an organization (such as a healthcare provider, service provider, sales organization, etc.) for the mutual benefit of both the organization and individual users. In the discussion below, these the organization is generally referred to as a “provider” “healthcare provider” or “healthcare facility”, and an individual user is referred to as “user” or “patient”. It will be understood that these references are used for convenience of describing the disclosed embodiments and are not intended to be limiting in any way.

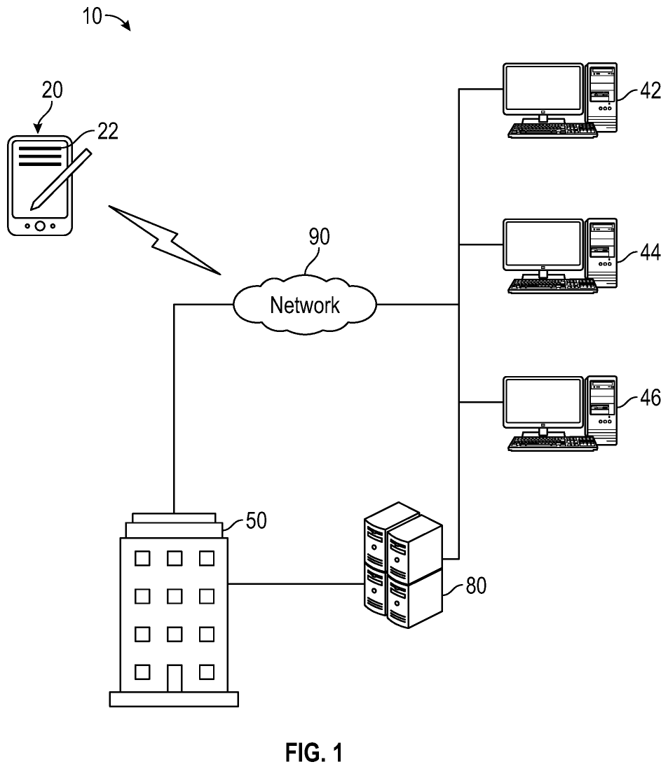

Turning now to FIG. 1, a general illustration of the systems and components of the wireless patient check-in system 10 is presented. As will be discussed below, the system generally coordinates and communicates between a patient mobile device 20, a healthcare facility 50 and a data center 80 (or computing center). In addition, these components will be able to communicate via a cloud-based communication system 90 using well recognized methodologies. Further, other computing devices, such as personal computers or terminals 42, 44, and 46 can also provide resources for user inputs. It will be appreciated that these systems can be housed or supported at different locations, and that different configurations are possible. For example, data center 80 may be located within healthcare facility 50 or could be hosted as part of a large-scale computing system. Further, mobile device 20 could take several well recognized forms such as a smartphone, tablet, laptop computer, smart watch, etc.

In the illustrated embodiment, the primary user interface for wireless check-in system 10 is a patient application 22 designed to run upon mobile device 20. More specifically, this involves a GPS enabled application which is installed on a patient's mobile device 20. While patient application 22 (also referred to as check-in application 22 or mobile application 22) provides several features, it does include the ability to obtain/retain the following information from patient:

Name, address, phone number

Photo Identification

Healthcare insurance card (or related information)

GPS based location data

Mobile application 22 also has the ability to store many different profiles corresponding to different people who may access healthcare or need to check-in via the mobile device 20 on which mobile application 22 runs. For example, a single mobile app (running on a single mobile device 20) may be able to retain information for all members of a family, thus allowing a parent to use the mobile app for their personal check-in, and for the coordinated check-in of their children or other family members.

Outlined below and illustrated in the various figures is one example of how mobile application 22 is used to sign-in and carry out various functions. It will be understood that mobile application 22 is coordinating with several related systems to carry out several of the features outlined above. As will be well recognized, initial steps will involve downloading the necessary mobile application 22, setting-up their account, and then interacting with the healthcare system in many ways. While the details may vary, the systems and processes outlined below further highlight the advantages and beneficial features involved with this embodiment of the wireless check-in system 10.

The following discussion also outlines the details of a system managed and supported by a single organization. It will also be understood that the invention is not limited to this implementation and may be supported by or coordinate with other systems.

As an initial step in the process, a user will download the appropriate application for use on their mobile device 20. There are multiple pathways for the user to find the appropriate download repository. These include but are not limited to: (i) a user may scan a QR code that is displayed in a provider office (i.e. hospital, clinic, therapist, or any other care facility) which links to a download source; (ii) the use of links to the download source are available on provider websites, Social Media (e.g. Facebook, Twitter, Instagram, etc.) and other related websites such as health insurance sites; (iii) a push notification enabled within an existing electronic medical record by which a user's mobile device receives a text message with a download link via an automated process; or (iv) searching existing sources such as the Apple App Store or Google Play. Clearly, mobile application 22 may be installed on mobile device 20 at most any point in time and at most any location (including while the user is in the provider facility). User validation and registration (described later in this document), would also be possible while the user is within the provider facility.

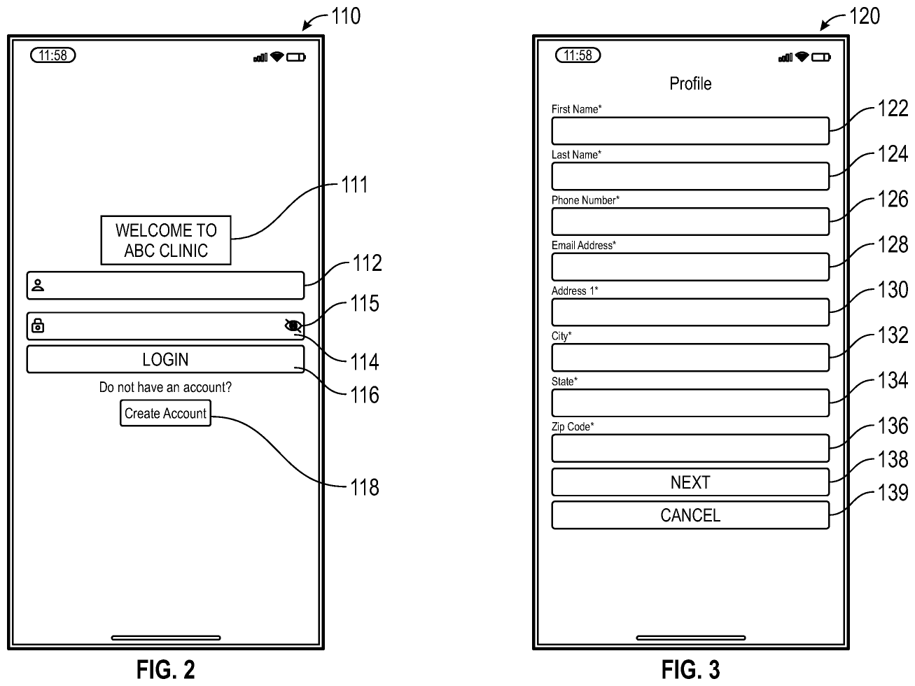

Turning now to FIG. 2, and exemplary user sign-in screen 110 is presented. This type of initial presentation will be expected by most users, as it includes several well-known features such as a welcome announcement 111, a username entry box 112, a password entry box 114 and a login button 116. This sign-in screen 110 also presents the user with the ability to display their password while typing by using a display tool 115. For use when initially setting up an account, the sign-in screen 110 also presents a create account button 118 which is used to launch additional functions.

When the create account button 118 is pressed or activated, a user profile screen 120 will be presented on mobile device 20, which is generally illustrated in FIG. 3. On this user profile screen 120, a user is presented with several data entry boxes, including a first name box 122, a last name box 124, a phone number box 126, an email address box 128, address line box 130, a city box 132, a state box 134 and a zip code box 136. Again, each of these boxes are set up to allow data entry from a patient using mobile device 20 using well understood methodologies. In addition, a user is presented with a next button 138 and a cancel button 139 with provides further navigation utilities. Each of these entry boxes are used to collect information from patients. While text data entry is shown in FIG. 3, it is understood that other data entry mechanisms are possible, such as drop-down menus, selection wheels, etc.

To provide further levels of security and to maintain the integrity of the system, a registration and validation process will occur at this point in the “create account” process. In one embodiment, the registration and validation process will operate on the user mobile device 20 within mobile application 22 as information is collected, but also coordinates with system servers. Generally, this registration and validation process involves a series of further user text inputs and photographs of user identifying documents. In one particular example, after entry of the user's email address a confirmation email will be sent to that email address allowing validation of this information. The email sent to that email address will present the user with a “confirm email address button” by clicking on this button, a message will be sent to the servers hosting the system, thereby confirming that the email address is valid and the mobile user has access to the email address that was entered during the registration process. Alternatively, several authenticator processes can be implemented, such as two factor authentication requiring the entry of a code or password that has been sent to the user via text message or email.

Similarly, the system will validate mobile phone numbers when the user inputs his or her mobile phone number. A text message will be generated by the system servers, which will send a validation code to the mobile number entered by the user. The user will confirm the validation code within mobile application 22 confirming that the user has access to the mobile number entered during the registration process.

At this point, the system has confirmed that a valid email address and mobile phone number have been entered. The user is then prompted to create a username and password using any preestablished rules that have been implemented. Upon entry of a proposed username, the application performs a data check to see if the username is already in use. If the username is already in use, the application will generate a “username already in use” message and the user will be prompted to create a different username.

As would be anticipated, the user is then prompted to create a password. Again, preestablished ruled may be implemented, such as requiring the password to be of sufficient length and to utilize upper case letters, lower case letters and symbols so as to decrease the likelihood of a bad actor obtaining the password by way of a brute-force scheme. Password parameters are set on the system servers and are changeable.

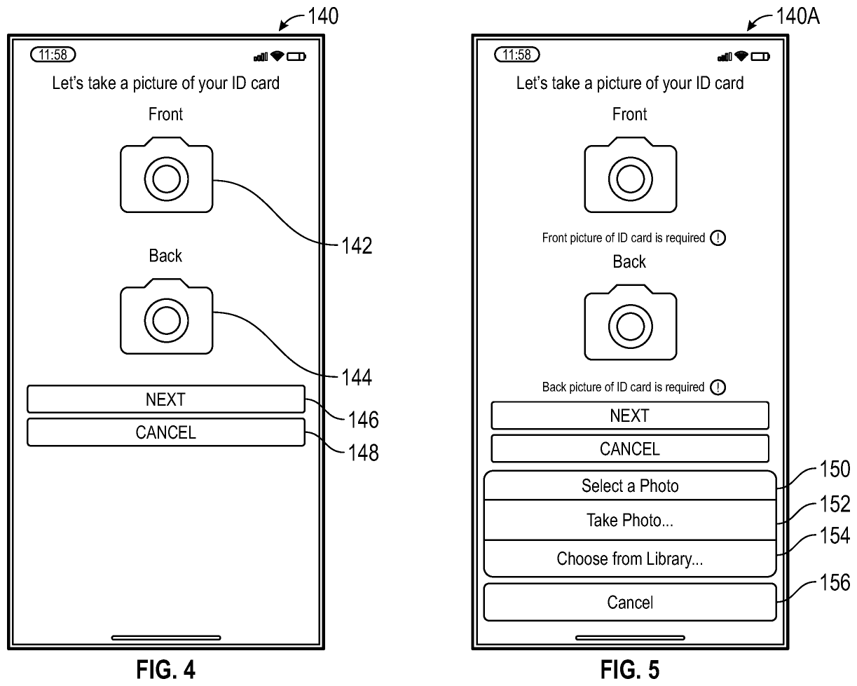

As a next step, mobile application 22 is then able to access the cameras on mobile device 20 to allow for the input of additional information. More specifically this enables mobile application 22 to take photos of certain documents and store those files within the application. These identifying documents could include: (i) a Driver's license/ID Card with photo (front and back); (ii) an Insurance Card (front and back); and/or (iii) any relevant medical records. Other information is clearly possible. One example of the interface screens presented to the user during this process are shown in FIGS. 4-6. In this example, a picture capture screen 140 is presented in FIG. 4, which provides a user with the ability to capture the front of an ID card using a front image button 142, and the ability to capture the back of an ID card using a rear image capture button 144. As shown, next button 146 and cancel button 148 are provided to allow appropriate navigation to a next step when this is appropriate.

Referring now to FIG. 5, capture screen 140A is shown, where the user has been provided with the ability to select existing photo using button 150, or to take a photo using selection button 152. Although not specifically illustrated, it will be recognized that these buttons will allow a user to obtain an appropriate image using necessary resources within mobile device 20. It will also be apparent that the user has the ability to navigate through other files on mobile device 20 using a Choose from Library button 154. If problems are encountered or things just don't look right, the user can simply cancel this process using cancel button 156.

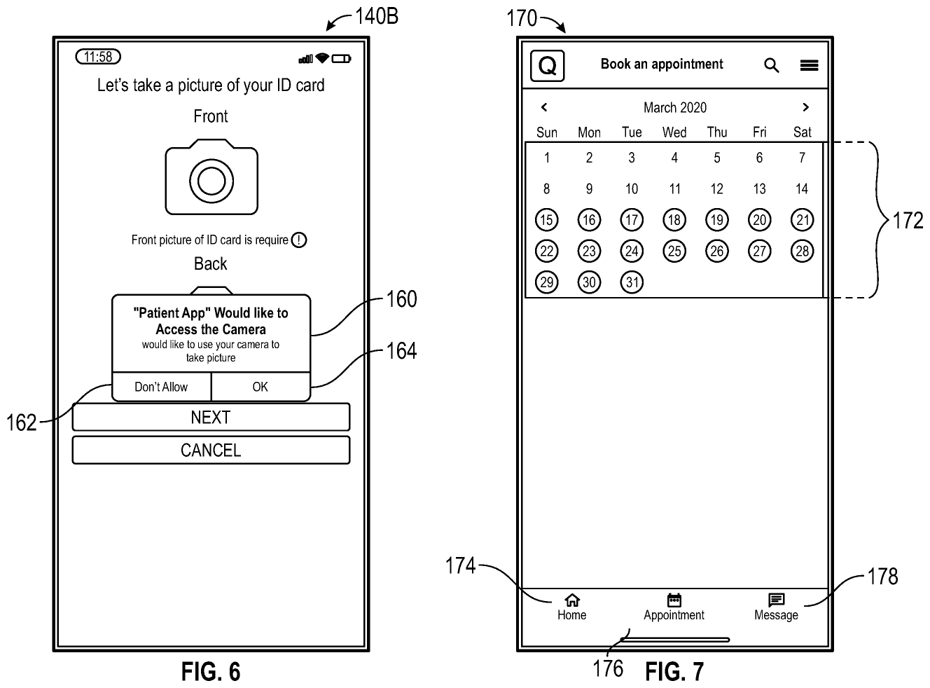

As mentioned above, the user will have the ability to make use of utilities on mobile device 20 to capture information, such as the camera and the voice recorder. FIG. 6 provides one illustration a further image capture screen 140B, where the camera tool is being initiated. In this image, a Camera Access Notice 160 is presented to the user, indicating that the application would like to access the camera. As shown, the user can approve or deny this request using OK button 164 or Don't Allow button 162. If approved, the application will launch an image capture screen (not shown) in a well understood manner. Similarly, a Don't Allow button 162 will cause the application to navigate back to previous screens.

As mentioned, these documents are stored within mobile application 22 and can be accessed as needed by other utilities within the system. It is contemplated that optical character recognition software within mobile application 22 will confirm that the document is legible, and that the name on the driver's license matches both the name on the insurance card and the name on the user profile. This provides another level of authentication for data entered by the user. If matches are detected, a “registration complete” confirmation will be sent to the user. If a match is not detected during the above-mentioned confirmation steps, an error message will be generated, and the user will be asked to re-enter the information. If a match cannot be obtained, the user will not be permitted to register within the application and will be prompted to contact the provider for instructions on how to proceed. It is contemplated that the provider could be permitted to enable a setting that allows a non-validated registration to be completed on the mobile device. Alternatively, a provider representative may be allowed to intervene and assist with registration. If the provider enables non-validated registration, the user will be presented with the “registration complete” message, but a flag will be sent to the provider indicating that the user data has not been validated. It will be then be the provider's responsibility to validate this data at the next encounter with this user.

Mobile application 22 can also prompt the user to take a picture of him or her self to provide additional levels of security and data protection. Facial recognition software allows the system servers to compare the picture of the user to the picture on the driver's license to ensure that the picture on the license matches the photo of the user. This function will help to reduce healthcare fraud by preventing an uninsured person to masquerade as an insured person in order to use the insured person's healthcare benefits for his or her self.

The registration process for minors is necessarily different because the minor may not have an email address or a driver's license/ID card. For person's under age 19, a check box is enabled next to the prompt for these photos (not shown in the Figures) which is marked “I don't have one.” If the user does check these boxes, the data and photo validation processes described above are disabled and the user is permitted to complete the registration process without the data validation check.

In the case of a minor, a similar flag is set on the provider screen alerting the provider to the fact that data validation was not possible. It will be the responsibility of the provider to validate the data in this instance.

Once set-up has been achieved, the user has the ability to book an appointment through mobile application 22, using additional scheduling tools. Examples of the screens used to schedule appointments are presented in FIGS. 7-10 and are discussed in further detail below. Again, the illustrated screens provide an example of how certain screens might look in one embodiment of the wireless check-in system 10.

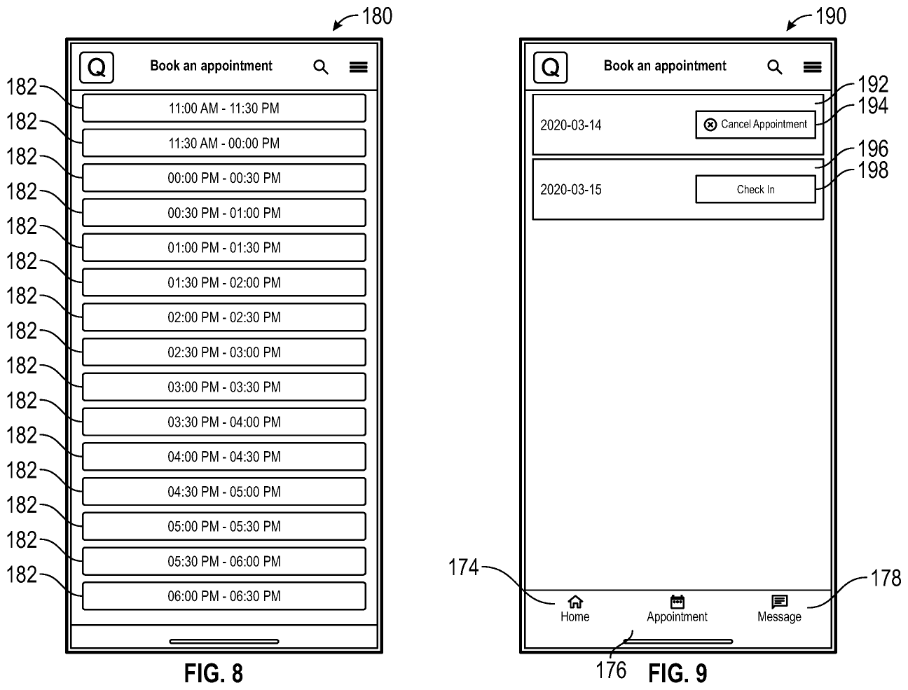

Turning now to FIG. 7, a calendar view selection screen 170 is presented. Here, an interactive calendar section 172 is presented to the user which allows for the selection of a particular day to book an appointment. When one of the dates on calendar section screen 172 is selected, a time select screen 180 is present to the user. An example of time select screen 180 is shown in FIG. 8, which show a plurality of time slot buttons 182. In each case, these time slot buttons 182 can be selected by a user, thereby allowing for the selection of the desired appointment time.

Referring again to FIG. 7, a user is presented with navigation buttons at the bottom of calendar view screen 170. In this embodiment, these include a home button 174, an appointment button 176 and a message button 178. Those skilled in the art will recognize that these navigation buttons will allow for navigation to other screens within the application. Although these navigation buttons are presented as part of the calendar view screen 170, similar utilities can be placed on several of the screens presented during operation of the application.

Returning to the functionality of check-in application 22 design to run on mobile device 20, there is also the ability to manage appointments that have been scheduled. FIG. 9 shows an appointment screen 190 which presents information to a user regarding scheduled appointments.

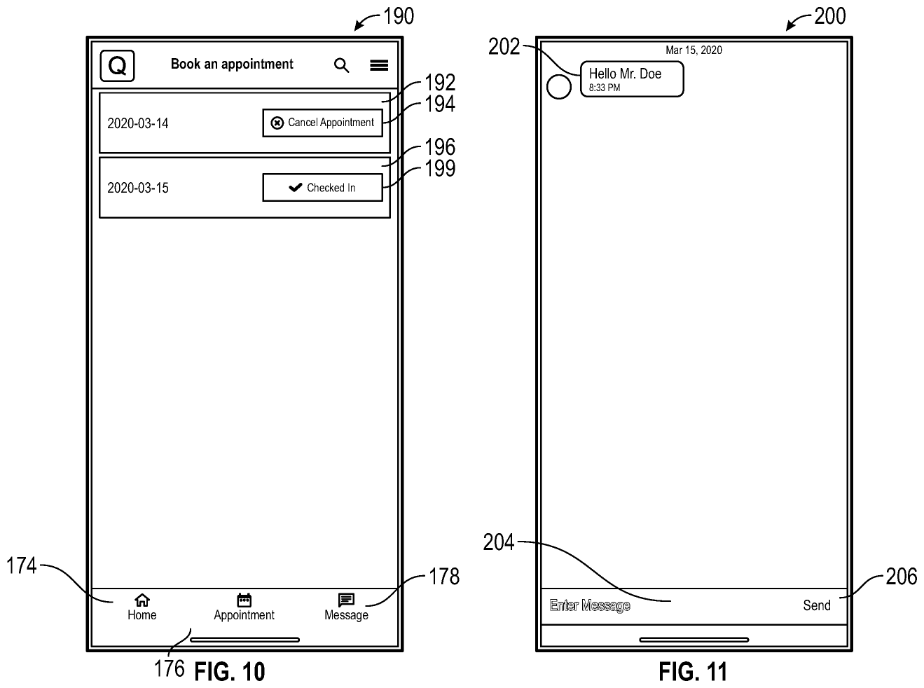

In this screen, a first appointment box 192 and a second appointment box 196 presented are presented to the user. In first appointment box 192, a cancel appointment button 194 allows a user to cancel related appointments. Alternatively, second appointment box 196 includes a check-in button 198. As would be anticipated, check-in button 198 allow a user to check-in for scheduled appointments. After activating check-in button 198, this will change to an appointment status indicator 199 to “Checked In” as shown in FIG. 10.

In many circumstances it may be necessary to contact or communicate with the patient regarding an upcoming appointment, to request information, or to simply confirm certain details regarding an upcoming appointment. For these situations, the system has a various communication features that can be used, including a text messaging feature and additional questionnaire features. Although this is very well recognized, one example of a messaging screen 200 is shown in FIG. 11. As illustrated, a text message bubble 202 is presented to the user at an upper portion of messaging screen 200, and a text entry box 204 is presented at a lower portion. Also available to the user is a send button 206 for use when messages are composed and ready to send.

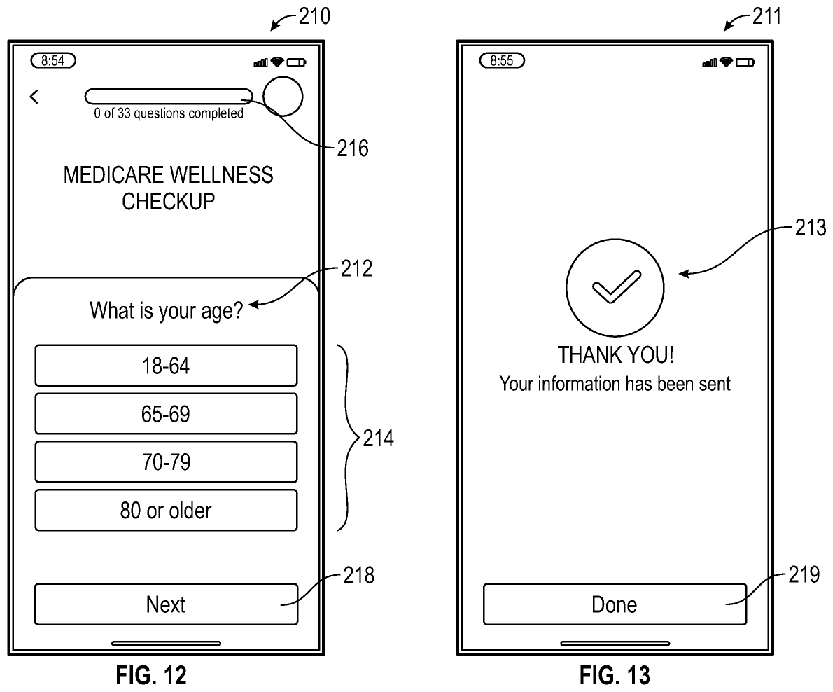

In addition to the functions provided in the messaging screen, check-in system 10 provides the ability to exchange additional information. During the actual check-in process, the above-mentioned mobile text messaging within the application can allow the user to communicate directly with a relevant department or group within the healthcare system (e.g., provider, scheduler or nurse). This will further allow the patient to supply additional information, such as symptoms, conditions, or other data which may be relevant. In many cases, this may include the presentation of specialized screens to the user, will have specific questions, text entry boxes, drawing tools, rating scales, etc. Further, there may be many instances where the healthcare provider may to send or push a questionnaire to the patient via the patient mobile application 22. For example, it may be necessary for the patient to complete a Medicaid questionnaire prior to the scheduled appointment. Using the existing communication tools, this questionnaire could be provided to the patient in various formats, and a completed questionnaire could similarly be received. One example of this capability is generally illustrated in FIGS. 12 & 13, wherein a questionnaire is pushed out to the patient prior to their appointment. More specifically, FIG. 12 shows the presentation of a questionnaire screen 210, which provides the start of a Medicare Wellness Checkup form. In this example, a number of questionnaire screens will be presented to the patient as they progress through the question process. Here, the exemplary questionnaire screen 210 is the initial question presented to the patient. A question presentation 212 is centrally located on the screen, followed by a number of selection boxes 214. To track status, a status indicator 216 is presented at an upper portion of questionnaire screen 210 which highlights how many questions will be involved and how many questions have been completed. Once a particular question has been completed, the patient will use a “Next” button 218 to move on to the next question. As the patient progresses through the questionnaire, subsequent questions will be presented, with each providing a data entry mechanism of some type which could include selection boxes, data entry boxes, rating scales, etc. After completion of the questionnaire, a confirmation screen 211 is presented to the patient which confirms that the questionnaire has been completed and the information has been sent to the health care provider. An example of this confirmation is presented in FIG. 13 which show a message presentation 213 and a final “Done” button 219 to move on to the next step of the overall check-in process.

Based upon information received through the above-mentioned text communication or any questionnaires pushed to the patient, it may also be necessary for the healthcare provider to call a patient and further investigate severity of the patient's condition. Additionally, the text messaging capabilities will allow the patient to get instructions (e.g. go to lab or X-ray or wait in your vehicle for further notice) or fill out necessary questionnaires (e.g. family history, travel history, etc.). This illustrates how many of the historical check-in tasks are completed independently by the patient, and at a point in time before the patient enters the facility.

In many cases where information is exchanged, a confirmation process within the mobile application 22 allows the patient to securely send identifying documents, insurance payment information, other health records, personal information, and other sensitive data to the healthcare provider in a secure manner in order to complete the check-in process. Generally, when information is being exchanged, the check-in system will coordinate with elements of the mobile application 22 to provide levels of back-end security. For example, the mobile application 22 and the check-in system will both include utilities that will generate predetermined trigger sequences which will precede the transmission of information. These trigger sequences confirm that the related information is valid and is coming from a verified source. These trigger sequences can also be used in addition to many well-known encryption techniques to maintain the confidentiality of information. In addition, the mobile application 22 and check-in system 10 can make use of GPS based location data to serve as a double check and in appropriate instances to confirm that the person checking in is actually in an expected physical location.

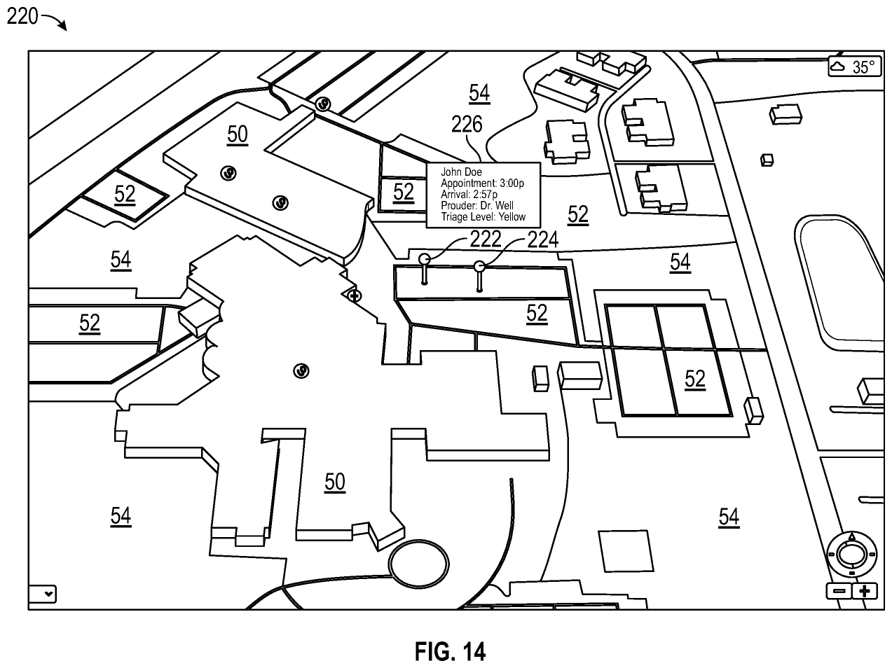

As is apparent from the discussion above, it is necessary for check-in application 22 to interact with the check-in system and/or the healthcare provider systems which may be running on a remote server, in the cloud, or on a local computing system at the provider facility. As an additional benefit of the interaction provided by wireless check-in system 10, parking lot/outdoor triage of patients is possible as they arrive at the provider facility. This is particularly useful in situations where the location is overwhelmed by patient volume, or where patients are likely to be contagious with an infectious agent. Further, this unique process also allows physical separation of patients outside of the facility while the application sends data to the facility indicating patient location, time of arrival, severity of illness. Information related to patients located within the area can be presented to the healthcare providers in an interactive graphical format, such as the map view 220 illustrated in FIG. 14. This allows healthcare providers to easily see the number and location of patients and allows them to easily pull additional information as desired. In this example, patients are represented by indicators such as pins, and information about triage levels is easily visible. It is possible to have triage levels which are presented with varying graphical indicators, such as different colors (e.g. a green level (walking well and generally mobile), a yellow level (will require medical attention), and a red level (will require immediate attention)). In addition, varying levels of information can be presented, such as the time of last communication along with current waiting time. Additional information could also be pulled up by clicking on a particular pin.

Map view 220 of FIG. 14 presents a geographical representation of the healthcare facility 50, area parking lots 52 and facility grounds 54. Generally, this data presentation (i.e. map view 220) of the healthcare location provides a “tracking board” or “patient status map” which identifies checked-in patients in the area and ensures that patients are attended to. In this presentation, two such patients are shown as a first pin 222 and a second pin 224. In addition, a data bubble 226 is shown which presents information related to first pin 222 (i.e. a first patient). Additional patient information could be presented, which might include appointment time, how long patient has been in outdoor triage area, an indicator if patient has not communicated with healthcare provider for a specified period of time, and indicator if patient has not left healthcare location after a specified period of time.

It is noted that patient status information can be provided in various formats, such as a 2D tracking board or patient status map 240 as shown in FIG. 16. This type of display gives healthcare providers extremely valuable information and allows for the handling of patients according to their locations and initial triage (which has been achieved via previous information exchanged). Importantly, this insures that no patients are missed, lost or overlooked.

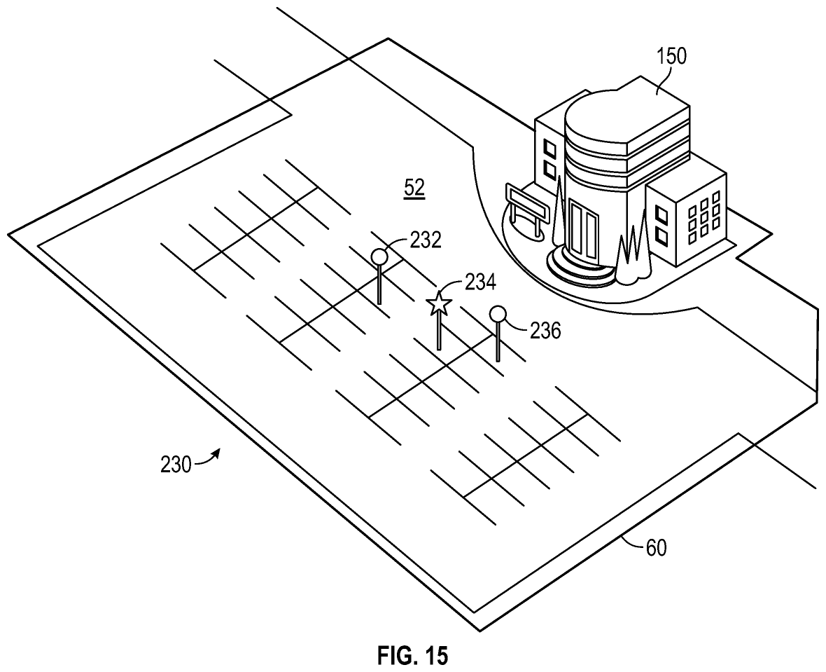

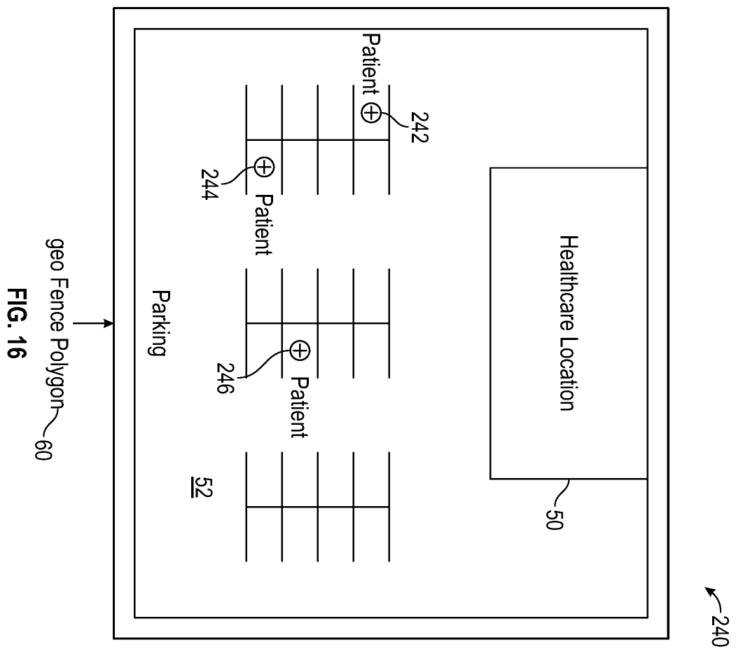

One additional feature provided by the mobile application 22 is the ability to establish and use a geofence around all provider facilities (e.g. clinics, hospitals, medical offices, etc.) and to determine if a patient has entered this area. Using GPS sensors within mobile device 20, the system can automatically prompt a patient to check-in for an appointment when the mobile device enters a provider facility geofenced area. FIG. 16 shows one example of geofence 60 as provided in the 2D tracking board 240. Additionally, FIG. 15 provides a second map view 230 of healthcare facility 50 and shows a defined geofence 60. In this embodiment, geofence 60 is configured to surround healthcare facility 50. Whenever a user enters this area, check-in system 10 can capture this information and provide appropriate notices to both the user and the healthcare provider. For example, when a patient enters parking lot 52, information is automatically sent to provider systems, which will then cause information about this patient to appear on map view 230. In this example, a first patient 232, a second patient 234 and a third patient 236 are shown. The addition of geofence 60 allows for the combination of GPS location information along with user identifying documents to enable a patient to check-in for an healthcare visit. It is noted that the patient “pins” shown in FIG. 15 are represented with different shapes, thus providing an indication of different priority or triage levels (e.g. second patient 234 is shown with a “star” at the top of the illustrated pin).

Although not specifically illustrated, the check-in application 22 running on the patient's mobile device 20 can also provide additional mechanisms to communication further information. For example, the patient may have the ability to notify the healthcare provider they are in transit to the healthcare facility 50 for a scheduled appointment. To achieve this, one embodiment may provide a reminder to the patient that an appointment is upcoming. As part of that reminder, a response option could be provided to the patient, (such as another button) to signal “I′m in route to facility.” In those circumstances where a patient has a scheduled appointment at a healthcare facility 50 and they do not notify the healthcare provider, they will get an appointment reminder a specified duration before the appointment through the patient application. The patient also has the ability to communicate their status, such as those cases where they are in distress and will be in need of immediate services.

As discussed in detail above, the check-in system makes use of GPS data for various purposes. As another example, when the patient is en-route to healthcare location, check-in system 10 it will update healthcare provider regarding the patient's GPS coordinates during specified intervals.

Wireless check-in system 10 also provides the unique ability to stage patients and do initial levels of triage at a location outside the provider location. For example, if the healthcare provider is trialing patients in alternative outdoor areas, such as parking lots, check-in system 10 will provide directions to the outdoor location via a real-time map with directions to the Patient application. Further, the patient application running on mobile device 20 will allow the healthcare provider to receive a notification indicating that the patient is in a designated outdoor triage area.

In those situations where outdoor patient triage is being used, the patient application will be notified by the healthcare provider application they are in a “Holding” state and that the patient is to remain in the outdoor triage area until further notice. To deal with more critical situations, patient application will have a “Panic” feature to notify healthcare provider application that Patient is in need of immediate attention. Patient application 12 will also have a feature to notify the healthcare provider application that the patient is in need of help transferring into the healthcare facilities.

At an appropriate time, patient application 12 will be notified by the healthcare location application when the providers are ready to see the patient, and the patient is allowed access to the healthcare facility. Patient application 12 will also prompt the patient to confirm that they are moving from the outdoor triage area to the facility, as instructed, and will notify the healthcare provider application when patient has entered the facility.

Healthcare provider application can also identify when a patient is transferring from the internal facility back to the outdoor triage location, an is to wait for further steps (i.e. a subsequent appointment, lab results, etc.). Patient application 22 continues to periodically update healthcare provider application regarding location. If discharged patient fails to leave identified outdoor area within a specified period of time, healthcare location staff are notified. Similarly, if a patient leaves prematurely, the healthcare provider is also notified.

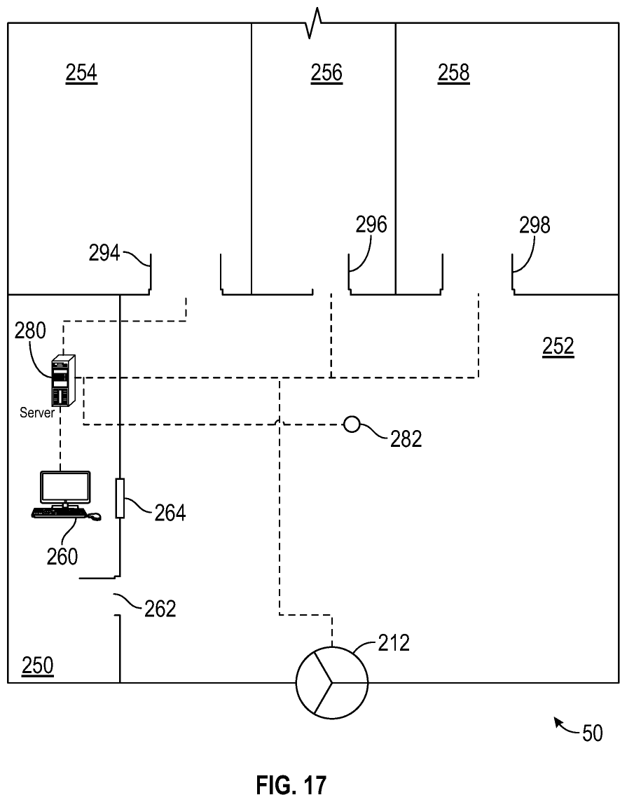

In addition to the notification and communication outlined above, wireless patient check-in system 10 can provide the capability to automatically guide the patient into healthcare facility 50. FIG. 17 presents one exemplary and simplified view of healthcare facility 50, which has an attendant room 250, a main lobby area 252, a treatment room 254, a lab 256 and an exam room 258. It is contemplated that an attendant could be stationed within attendant room 250 and would have access to the provider information outlined above using terminal 260 (i.e. would have access to information related to schedules, appointments, and the above mentioned map views). An access door 262 and a window 264 could provide the ability of patient to communicate with the attendant, if necessary. A facility server 280 could also be housed within attendant room 250, which would provide easy access to necessary information. Although shown in this location here, facility server could be located at data center 80, could be part of a cloud-based computing system, or could be structured differently.

To provide additional capabilities, facility server 280 is in communication with main entry door 292 (illustrated here as a revolving door), treatment room door 294, lab door 296, exam room door 298 and an area sensor 282. With these components, wireless check-in system 10 would thus be able to track a patient as they entered facility 50, and open appropriate doors to provide access to necessary areas. For example, if the patient is instructed to go to lab 256 for tests, check-in system 10 can sense when the patient is close to lab door 296, and automatically open it at an appropriate time. Although not shown, it is further contemplated that displays could be placed throughout facility 50 which would be configured to provide notices and instructions. For example, a display outside lab 256 could present a patient with a “welcome” message, when they are detected in close proximity to door 296.

In practice, it is also possible that alternative techniques could be used to provide door access. In another embodiment, patient application 22 would serve as a key to opening facility doors, transmitting appropriate digital keys at the necessary times. In this situation, patient application 22 could provide the patient with a display screen asking for confirmation that they are interested in opening a prescribed door. Upon such confirmation, patient application 22 would then transmit a digital key or code that would be recognized by a door access mechanism. This transmission could be achieved via Bluetooth signals, or via other wireless communication techniques. Alternatively, it is possible for the medical facility system to send a barcode to the patient via the chat feature. The barcode would be delivered over a secure channel, thus providing an additional level of security. The patient would then scan the barcode at a scanner located near the necessary door, thus granting him or her access to a particular room or the building itself.

In each case, additional levels of security are achieved because the receiver device controls the door lock and does not allow entry unless proper verification is completed. Upon receiving a signal containing then proper verification data from a mobile device (via Bluetooth, chat, scanner, or other data entry techniques) the receiver device would open a door or send a message to another organization authorizing release of information needed to gain entry.

In order to provide the functionality discussed above, check-in system 10 coordinates the operation of various components to allow the necessary sharing of information. From a system architecture perspective, it is anticipated that these components will include a back-end system, the above discussed check-in application 22, a tracking application and a transformation layer. Details regarding each of these components are discuss below.

Back End System:

A cloud-based application is provided, which includes all code necessary for typical back-end processing steps and enables a provider (e.g. a healthcare provider or clinic) to coordinate the sharing of information as needed. This cloud-based application typically resides on and is executed from a central server(s), and further has the capability to display results on the provider's screen. Alternatively, the provider's devices may also execute code depending on the conditions of the request and functionality that is required. In this particular embodiment, the back-end system (or application) is accessible from a suitable internet connected device, thus providing additional flexibility. Most importantly, the back-end system is used by a provider business to interact with the mobile device user, and thus further coordinate the check-in process.

As previously discussed, when using the above-mentioned mobile application 22, a mobile device user (i.e. patient) is presented with a “Check-In” button 198 at a particular point in the process. When the patient presses the “Check-In” button 198 within the mobile application 22, data from the mobile application 22 (including GPS location, name, phone number, and identifying documents) are sent by way of the transformation layer (described in further detail below) to the back end system. Providers using the back-end system can use this data to complete the check-in process, which will likely include the solicitation of additional information. The back end system also supports multiple providers working at the same time, can accommodate multiple patients checking in at the same time and simultaneously displays real time check-in information from multiple mobile users. Each provider business will utilize its own back end application, which is provided with appropriate levels of security and data protection to avoid any data breach issues. The back end application is configured to support the common needs of many provider businesses, while also being tailored to meet any specific needs of the particular provider business.

Tracking Application:

As discussed above, the use of GPS location data provides specific functionality to check-in system 10. In addition, a tracking application is a software application which receives GPS location data from the various patient mobile devices and is then is capable of storing and displaying this location data in different ways for the purposes of data analysis. It is anticipated that this tracking application is more general than the above discussed location confirmation or geofencing capabilities discussed above. The primary use of the tracking application is to analyze disease spread in real time or by history for the purposes of predicting future spread, or evaluating the extent of current spread of disease. The tracking application can display a map and place individuals on that map in real time or by history. It can also analyze movement and interaction between different users and may be used to track the physical distance between users at different points in time. As needed, the tracking application also has the ability to export data to be analyzed by other software applications.

The tracking application is enabled by default. Patients are able to opt-out in the settings screen. The tracking application does have the capability to be set to always on in the event that a government entity legally requires that this function is enabled. The primary purpose of the tracking application is data analysis for research purposes. Specific measures will be included to manage data and to insure confidentially as necessary for the particular circumstances.

Transformation Layer:

To further facilitate coordination of the various systems and/or applications, a transformation layer provides a platform to relay data from the mobile application 22 to the tracking application and the back-end system. The transformation layer is a software platform that supports multiple simultaneous data inputs and outputs at the same time. For example two mobile users (i.e. patients) may present for check-in at two different providers. The transformation layer takes appropriate action to direct mobile user data to the correct back end provider. The tracking layer is massively scalable and can support thousands of simultaneous transactions.

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1. An automated system for managing an initial interaction between a healthcare provider and a patient, the system comprising:
a check-in application running on a patient mobile device, the check-in application configured to present information to the patient via a user interface hosted by the mobile device and configured to send and receive information; and
a computing system configured to send information to the mobile device and to receive information from the mobile device, the computing system having preexisting patient information which will accommodate the secure communication of information between the check-in application and the provider application, the computing system further running a provider application configured to coordinate a check-in process which comprises the collection and sharing of information between the check-in application and the provider application to achieve check-in for an existing appointment, wherein the check-in process comprises:
presenting information to the patient identifying the existing appointment and allowing the patient to advance the check-in process via the mobile device, wherein advancing the check-in process causes signals to be sent to the provider application indicating that the patient is interested in checking in and providing information regarding the location of the patient mobile device;
the provider application reviewing the preexisting patient information to confirm the identity of the patient and a plurality of details related to the upcoming appointment along with a patient record stored within the computing system to determine if the patient record includes all information necessary for the existing appointment, wherein the information necessary for the existing appointment includes a triage indicator, and, if additional information is necessary, sending signals to the check-in application to allow the patient to provide the additional information from the patient, the provider application thereafter receiving the additional information from the patient and saving the additional information as part of the patient record;
confirming that the the patient record includes the necessary information, the provider application reviewing the location of the mobile device, generating a tracking board for display to the provider, and sending signal to the patient via the check-in application with a patient instruction, wherein the patient instruction comprise at least one of a wait at location instruction, proceed to facility instruction, or proceed to alternative location instruction;
wherein the tracking board provides a graphical illustration of the patient location and the triage indicator based upon the necessary information.

2. The automated system of claim 1 wherein the necessary information comprises patient identification information, health insurance information, patient health history information, and current condition information, and wherein the current condition information comprises an indication of the severity of the current condition and is used to develop the triage indicator.

3. The automated system of claim 2 wherein the necessary information includes a completed Medicaid questionnaire that was pushed to the mobile device by the computing system.

4. The automated system of claim 1 wherein the check-in application and the provider application maintain communication so that the check-in process is initiated when the patient is a predetermined distance from the healthcare facility.

5. The automated system of claim 4 wherein the provider application maintains a geofence surrounding the healthcare facility and the check-in process is initiated when the patient mobile device is within the geofence.

6. The automated system of claim 1 wherein the communication between the check-in application and the provider application is secured using trigger sequences as part of the messages making up the communication.

7. The automated system of claim 1 wherein additional information is received via a questionnaire that is pushed to the patient, wherein the questionnaire comprises a plurality of questions that can be answered via the mobile devise user interface.

8. The automated system of claim 7 wherein the questionnaire pushed to the patient is used to determine the triage indicator.

9. The automated system of claim 7 wherein the provider application maintains a geofence surrounding the healthcare facility and the check-in process includes an indicator to the computing system whenever the patient mobile device is within the geofence.

10. A mobile check-in application stored within memory of a mobile device associated with a patient and configured to coordinate the operation of a plurality of modules on the mobile device, the modules comprising:
a setup module allowing the patient to enter patient information comprising general patient data, insurance data, and health condition data;
a scheduling module to accommodate communication with a healthcare provider computing system allow the scheduling and management of appointments for healthcare services; and
an automated check-in module which monitors a GPS location of the mobile device and a GPS location of the healthcare facility to thereby initiate a check-in process when the GPS location information confirms that the patient is at a healthcare provider location, wherein the module further communicates with the healthcare provider system to carry out a check-in process, wherein the check-in process allows for the confirmation of an appointment, the transmission of patient information to the healthcare provider system, and the receipt of check-in instructions, wherein the patient information comprises a patient identifier and GPS location data sufficient to allow the healthcare provider to project the patient information onto a graphical map display.

11. The mobile check-in application of claim 10 wherein the transmission of information to the healthcare provider comprises the transmission of the general patient data, insurance data, and health condition data, and establishes a patient triage record for use by the provider system.

12. The mobile check-in application of claim 10 wherein the patient information transmitted to the healthcare provider comprises a triage indicator, a patient identifier, health insurance information, and patient health history information.

13. The mobile check-in application of claim 12 wherein the mobile application is configured to receive a questionnaire that is pushed to the mobile device from the healthcare provider, to complete the questionnaire using the user interface and to transmit the completed questionnaire information back to the healthcare provider.

14. The mobile check-in application of claim 13 further configured to receive a signal from the healthcare provider indicating that mobile device is within a geofenced area defined by the healthcare provider computing system and thereby launch the check-in application.

15. The mobile check-in application of claim 14 wherein the scheduling module and the check-in module comprise text message capabilities thereby allowing the patient and the healthcare provider system to exchange information via text message.

16. A patient appointment application stored and running on a healthcare facility computing system which allows a healthcare provider to manage and coordinate appointments via communication with a patient mobile device, the patient appointment application comprising:
a communication module configured to securely send messages to and securely receive messages from the patient mobile device;
a scheduling module to manage appointments at the healthcare facility, the scheduling module further configured to communicate via the communication module with the patient mobile device to schedule and confirm appointments;
a check-in module configured to communicate with the patient mobile device to carry out a check-in process, wherein the check-in process comprises the patient appointment application determining the existence an upcoming appointment and communicating with the patient mobile device thereby allowing the patient to check-in, the check-in process further configured to receive a GPS location signal from the patient mobile device indicative of the patient location and patient status information indicative of a the patient condition; and
a provider display module configured to generate a graphical map display for the healthcare provider of all checked-in patients within a predetermined area, the graphical map display providing an interactive representation of the location of the checked-in patients within the predetermined area based upon the received GPS information, and providing the ability for the healthcare provider with the ability to view and retrieve patient identifying information and patient status information, and to provide further instructions to the checked-in patients.

17. The patient appointment application of claim 16 wherein the patient status information can be used to allow the healthcare provider to assign a triage level for each check-in patient, and wherein the provider display module will present a graphical indicator of the triage level.

18. The patient appointment application of claim 16 wherein the check-in module is further configured to solicit information by pushing a questionnaire to the patient mobile device for completion by the patient.

19. The patient appointment application of claim 18 wherein the questionnaire is a medicaid questionnaire.
